(19) United States
(12) Patent Application Publication
(10) Pub. No.: US 2026/0219170 A1
(43) Pub. Date: Jul. 30, 2026

(54) SYSTEM AND METHOD FOR ESTIMATING SPECTRAL RESIDUAL VIABILITY OF A FOOD PRODUCT

(71) Applicants: SOUTH EAST TECHNOLOGICAL UNIVERSITY, Waterford (IE); UNIVERSITY COLLEGE DUBLIN, Dublin (IE)

(72) Inventors: Ultan MCCARTHY, Waterford (IE); Anastasia KTENIOUDAKI, Dublin (IE); Jean Pierre EMOND, Tampa, FL (US); Ricardo BADIA-MELIS, Madrid (ES); Colm O'DONNELL, Dublin (IE); Carlos Alberto Esquerre Fernandez, Waterford (IE); Maria Cecilia DO NASCIMENTO NUNES, Lisboa (PT)

(73) Assignees: SOUTH EAST TECHNOLOGICAL UNIVERSITY, Waterford (IE); UNIVERSITY COLLEGE DUBLIN, Dublin (IE)

(21) Appl. No.: 19/145,477

(22) PCT Filed: Jan. 22, 2024
(86) PCT No.: PCT/EP2024/051454

§ 371 Date: Jul. 2, 2025

(30) Foreign Application Priority Data
Jan. 20, 2023 (GB) 2300861.8

(51) Int. Cl. G01N 21/27; G01N 21/17; G01N 33/02; G06Q 10/087; G06T 7/00; G06T 7/90

(52) U.S. Cl. CPC G01N 21/27; G01N 33/02; G06Q 10/08776; G06T 7/0012; G06T 7/90; G01N 2021/1765; G06T 2207/10024; G06T 2207/20081; G06T 2207/30004; G06T 2207/30188

(57) ABSTRACT

Disclosed is a system and method for assessing spectral residual viability (SRV) and corresponding remaining shelf life (RSL) of a food product, comprising capturing one or more images of the food product by a camera; analysing the one or more images to perform a visual assessment of a plurality of individual attributes of the food product, using respective plurality of vectoral algorithms; estimating a Quality Index (QI) of the food product based on an average summation of each individual analysis of each individual IE attribute; receiving a future storage temperature at which the food product is stored or transported; estimating a SRV and corresponding RSL of the food product based on the QI and the future storage temperature. The present invention provides a system and method that has the functional capability to merge both biologic product specific degradation models with user defined predicted consequence to predict SRV and corresponding RSL of the product.

### FIELD

The present disclosure relates to assessing food quality of a food product, and more particularly to estimating spectral residual viability (SRV) and corresponding remaining shelf life (RSL) of the food product.

### BACKGROUND

Current global food supply chains are not “resource efficient”, with fresh produce chains commercially operating at approximately 50% efficiency. This waste level has a devasting impact on food security affecting all people regardless of geography or social groupings. Coupled with this is the fact that currently anywhere between 30 and 50% of food produced is never consumed and inevitably goes to waste with negative impacts on natural resources and major economic losses globally. A 56% gap exists between the amount of food available today and that which will be required by 2050 to feed our increasing global population. We live in a world that produces enough food to feed everyone, yet more than 113 million people across 53 countries experience acute hunger, while a further 143 million people are living in stressed conditions and on the verge of acute hunger.

A major challenge when addressing food waste involves the significant inefficiencies along the supply chain, and in particular the inability to couple the natural biologic spoilage (primarily linked to temperature and time) to business/stakeholder processes (logistics/storage/distribution). The nature, diversity, duration and management of supply chains vary considerable across the world. In an example, currently within the strawberry industry, product assessment is performed manually, and due to personal variation in assessment (due to light and/or graders personal preference) this process is very subjective. Hence, this leads to major discrepancies in grading with a net overall increase in incorrectly graded berries and increased unnecessary waste.

There are several technologies in the market today which have a localised impact and fail to provide a systems (joined up) solution approach. These current solutions are driven by inefficient existing business processes resulting in 30% to 50% waste.

Commercially, the current systems provide an estimation of shelf-life (presented in ‘Best before’ date) based on empirical information (average product shelf-life at a particular time of the year/from a particular cultivation system combined with subjective visual evaluation). These systems are preassigned to the product and also assumes “ideal” constant postharvest conditions to predict the pre-assigned ‘Best Before’ date and so they are non-reflective of “real life” supply chains. Such systems lack flexibility to accommodate any “real life” changes which are an essential component of modern-day global supply chains.

Further approaches include in-situ vision systems at production or at retail, supplemented with sensors tasked to perform quality classification. Current systems are both static and reactive and completely lack proactive functionality. Such systems are usually used for production line management and automatic product grading. As the name suggests, these systems are non-mobile, reactive in functionality and require significant investment, user training and time.

Other commercially available solutions include the use of sensors (NIR) to provide information on maturity levels and estimation of shelf-life of a variety of products. These systems are negatively disadvantaged by the fact that they are both static and reactive and completely lack proactive functionality.

Some other available solutions include use of wireless sensor networks which involve the deployment of a variety of product specific sensors to monitor the environmental conditions to which the product is exposed. However, significant efforts have taken place to use these technologies to replace FIFO systems with information rich FEFO based systems using IoT and wireless networking to track and monitor supply chain conditions and provide estimation of shelf-life through dynamic coding.

Dynamic pricing systems are reactive in their approach to food spoilage and waste. They base their algorithms on a predefined “product duration/best before/use by” scenario. These systems are non-accommodating in their approach to product stability and lack the ability to factor in system exceptions i.e., they are reactive in their approach.

Each of the technologies listed above only provide partial assessment of the product throughout the supply chain. Each solution bases its assessment on the product being at its “recommended handling conditions” at time of analysis and fails to factor in any previous mishandling of the product thus yielding inaccurate prediction outcomes. Further, such existing systems are architected on a mass defined numeric product “shelf life” as opposed to a biologic centric based system. This results in a system that is process centric, meaning, that all decisions being made are done so without considering the natural biologic variance within the product, rendering the system unable to address the issues of spoilage and waste. Further, existing technologies are architected on a time to destination FIFO based system which lacks any ability to consider any transit mishandling.

Many, if not all the current systems are fixed to a production line or are too expensive for the product in questions (strawberry/fresh produce/fast moving consumer goods/perishable goods). Food, as a general rule is a low variety low margin commodity and for this reason, there is little to no scope for infrastructural quality tools investment to address significant waste levels. A key barrier to technology acceptance/deployment within food supply chains is the cost. Further, many of the quality tools currently being deployed in the food industry require skilled users and a significant level of training and technically problematic to implement. The agri-food industry relies on seasonal/low skilled/casual employment across many global regions.

US 2020/0251229 relates generally to a system and method for monitoring and quality evaluation of perishable food items in quantitative terms. However, said system and method requires both image and sensory data to perform analysis and make a freshness level decision of food items. The system requires a real time feed of sensory and visual data to make assessment. This may require significant infrastructural investment, such as wireless and environmental sensing technology to perform the computations.

US 2022/0327685 relates to an evaluation system that can receive image data of produce and identify a type of the produce using machine learning techniques. The evaluation system records the volatile properties of the food type over time and uses the presence of volatile components to predict the peak ripeness of the food. The “peak ripeness” is a term to denote best time to harvest. In addition, this system also requires the assignment of a UID (obtained through a photo) for each product as a reference and cross reference. This UID may be an SKU, QR code, sticker, stamp or label attached to the product. However, said system does not address the problems of food waste because it is practically impossible to link “time to harvest” and “food waste” in the context of a supply chain.

In view of above, there is a need for a system and method that can address the issue of commercial waste and can also reduce the instances of “subjective” produce grading, both of which would individually and collectively address the issues of food waste. Such method and system should objectively evaluate quality in real-time and predict an accurate remaining shelf-life based on known scenarios and adjustable to user-based scenarios or breaches.

### SUMMARY

According to the invention there is provided, as set out in the appended claims, a method for assessing spectral residual viability (SRV) and corresponding remaining shelf life (RSL) of a food product, comprising: capturing one or more images of the food product by a camera; analysing the one or more images to perform an assessment of a plurality of individual interest elements (IE) of the food product, using respective plurality of vectoral algorithms, wherein IE are generated by removing non-interest element (NIE) from total element (TE) of the food product; estimating a Quality Index (QI) of the food product based on an average summation of each individual analysis of each individual attribute, wherein each individual attribute is estimated based on one or more IE of the image; receiving a future storage temperature at which the food product is stored or transported at a later time; and estimating the SRV and corresponding RSL of the food product based on the QI and the future storage temperature.

In one embodiment, the step of analysing an image comprises: applying an image mask on the image to remove non-interest elements (NIE) from total elements (TE) therefrom; transforming the image from an RGB base to CIELAB base to enhance interest-elements (IE) discrimination potential for image analysis, and accurately represent the perceived colour of images being analysed; using machine learning models to estimate an entropy, standard deviation and range values of the image for a plurality of individual attributes of the food product.

In one embodiment, each individual attribute is selected from colour, calyx wilting, shrivelling, bruising and physical damage, decay and mould.

In one embodiment the method comprises the step of: employing machine learning algorithms and product specific kinetics modelling providing the end user with a product specific fully digitized SRV and corresponding RSL based on expected storage conditions, and time/distance to final destination.

In one embodiment the method comprises the step of:
estimating the QI of the food product based on analysis of visual spectra of wavelength ranging from 380 nm to 750 nm and frequency ranging from 4 Hz to 7.5*1014 Hz of the one or more captured images.

In one embodiment the method comprises the step of: merging the QI with a variable order kinetic modelling database to generate the SRV and corresponding RSL based on the future storage temperature.

In one embodiment the method comprises the step of:
storing the one or more captured and analysed images in a database for querying/retrieval at a later date.

In another aspect of the present invention, there is provided a system for assessing spectral residual viability (SRV) and corresponding remaining shelf life (RSL) of a food product. The system includes a memory to store the one or more instructions, a processor to execute the one or more instructions to: capture one or more images of the food product by a camera; analyse the one or more images to perform an assessment of a plurality of individual interest elements (IE) of the food product, using respective plurality of vectoral algorithms, wherein IE are generated by removing non-interest elements (NIE) from total elements (TE) of the food product; estimate a Quality Index (QI) of the food product based on an average summation of each individual attribute, wherein each individual attribute is estimated based on one or more IE of the image; receive a future storage temperature at which the food product is going to be stored or transported at a later time; and estimate the SRV and corresponding RSL of the food product based on the QI and the future storage temperature.

In an embodiment of the present invention, the processor is further configured to: employ machine learning algorithms and product specific kinetics modelling providing the end user with a product specific fully digitized SRV and corresponding RSL based on expected storage conditions, and time/distance to final destination.

In an embodiment of the present invention, the processor is further configured to estimate the QI of the food product based on analysis of visual spectra of wavelength ranging from 380 nm to 750 nm and frequency ranging from 4 Hz to 7.5*10¹⁴ Hz of the one or more captured images.

In an embodiment of the present invention, the processor is further configured to merge the QI with a variable order kinetic modelling database to generate the SRV and corresponding RSL based on the future storage temperature.

In an embodiment of the present invention, the processor is further configured to store the one or more captured and analysed images in a database for querying/retrieval at a later date.

Various embodiments of the present invention provide a system and method that has the functional capability to merge both biologic product specific degradation models with user defined predicted consequence to predict SRV and corresponding RSL of the product. The proposed solution will provide a standardised real time end to end qualitative assessment of product based on biologic history and predict SRV and corresponding RSL based on user defined forward carriage. The SRV and corresponding RSL would provide capability to adopt a FEFO (first-expired-first-out) as opposed to a FIFO (first-in-first-out) based management strategy. The RSL is unique in its ability to provide a product specific SRV and corresponding RSL based on qualitative biologic-based assessment as opposed to the current systems which merely provide qualitative assessment based on assumed predefined environmental controls. This will ensure that all produce in the supply chain would reach its destination in a safe, fresh, and timely state, thus increasing its chances of reaching the consumer with a reasonable remaining shelf-life. Any food that would potentially spoil (due to routing errors and or delays or mishandling) will be quickly identified allowing it to be re-purposed (tinning/juicing/freezing etc) thus reducing the overall food waste. The present invention would identify where the “mishandling/breach” has occurred, thereby indirectly highlighting weak points in the supply chain “mis-handling”.

The estimation of SRV and corresponding RSL of a food product is a cutting-edge solution in food supply chain management designed to minimise food loss and waste. It is unconventional and disruptive in its approach through fusing explicit optical and chemometric information with tacit product specific biologic data and supply chain stakeholder knowledge. The SRV and corresponding RSL estimation system and method uses visible spectra for the biological assessment of a food type. Existing vision systems focus on one single aspect of the food (as per above size, presence/absence of ingredients and or the colour etc), whereas the present invention considers a number of aspects (classifier groups) to compute the SRV and corresponding RSL. It is the combination of the individual (vectoral) elements that make the present invention different as compared to existing techniques.

The SRV and corresponding RSL estimation system and method only requires a camera and logic processor and only uses the image (as input for logic building) for full analysis. It does not require anything additional such as sensory input and or infrastructural requirements such as external sensors, volatile analytical technology or any type/kind of assigned UID or SKU. The SRV and corresponding RSL estimation system requires images only from visible spectrum. Thus, the RSL estimation method does not require any infrastructural investment (wireless and environmental sensing technology) as all computation is done by simply taking a photo of the food product. This would reduce the volume of waste produce being shipped across the supply chain (as currently up to 50% of produce goes to waste). This estimation method is cost-effective as compared to existing static systems which can cost up to €25,000 per camera.

### BRIEF DESCRIPTION OF THE DRAWINGS

The invention will be more clearly understood from the following description of an embodiment thereof, given by way of example only, with reference to the accompanying drawings, in which: —

FIG. 1A illustrates a system for assessing food quality of a food product, in accordance with an embodiment of the present invention;

FIG. 1B illustrates a sequence of steps of a method for assessing food quality of a food product, in accordance with an embodiment of the present invention;

FIG. 2 illustrates the supply chain control points across which the system can obtain an image for) prediction of RSL of food products; and

FIGS. 3 and 4 are examples of the spectral outputs depicting where the visible spectra represent the area of interest in the captured one or more images of strawberries.

### DETAILED DESCRIPTION OF DRAWINGS

The invention is not limited to the embodiments hereinbefore described but may be varied in both construction and detail.

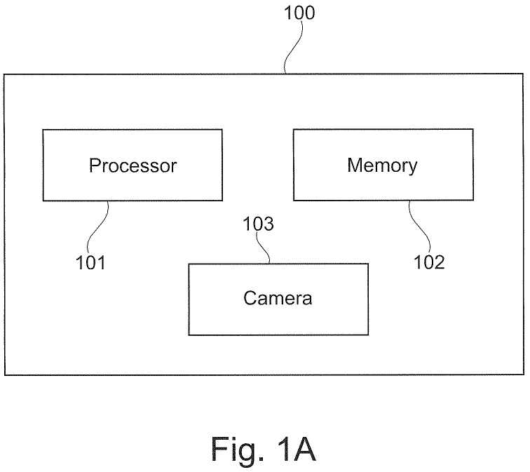

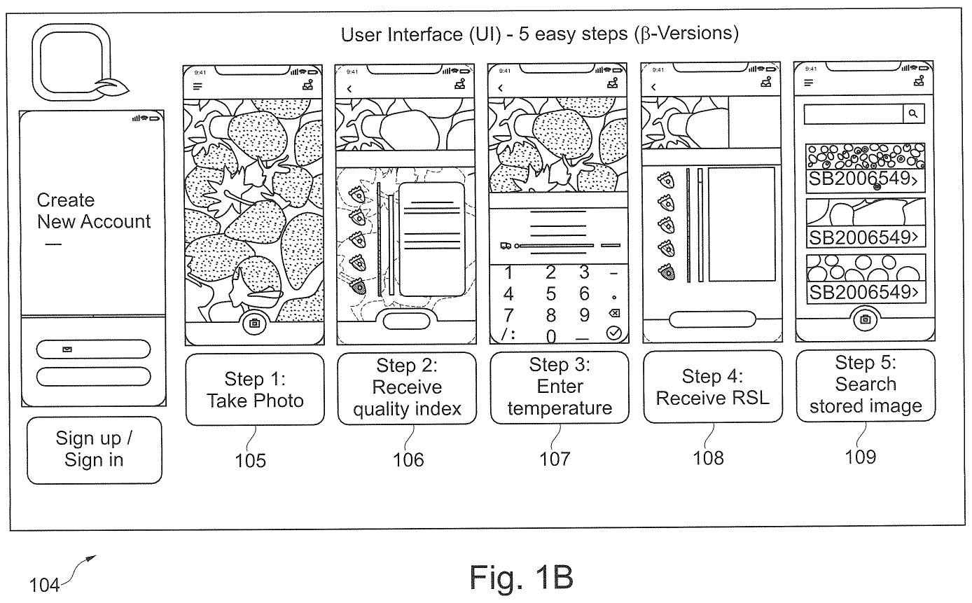

FIG. 1A illustrates a system 100 for assessing spectral residual viability (SRV) and corresponding remaining shelf life (RSL) of a food product, in accordance with an embodiment of the present invention. The system 100 includes a processor 101, a memory 102, and a camera 103. In an embodiment of the present invention, the processor 101, the memory 102, and the camera 103 may be part of a single computing device. In another embodiment of the present invention, the memory 102 and the camera 103 may be part of a single computing device, and the processor 101 may be implemented through a combination of computing device and a remote server communicatively coupled to the computing device.

Examples of the computing device include, but are not limited to, phone, tablet, computer, and PDA, or any other computing device that can be installed in weighing scales in a retail outlet or in CCTV systems in shops. This would allow the user to be based at any/each point across the supply chain (i.e. farmer, distributer, truck driver, logistics manager, retailer, shopkeeper, inspector, buyer/seller etc) to assess food quality of food product. It can be used in a field/in a shop/in a truck (i.e. all places a modern day mobile phone can work) and provide full functionality at each/every point for all stakeholders including trading partners and standards agencies. This would allow any user of the technology to use the application on their existing systems, as there is no specific hardware associated with it.

The system 100 implements a method 104 of assessing SRV and corresponding RSL of a food product. The method 104 has been explained in detail with reference to FIG. 1B.

Referring to FIG. 1B, at step 105, one or more images of the food product is captured by the camera 103. The system 100 may run an application that enables the camera 103 for capturing an image of the food product, and enables the processor 101 to perform analysis of the captured image. In an embodiment of the present invention, Vision Optical System (VoS) is provided in a computing device to enable the computing device to capture an image of the food product. It is to be noted that images of the food product can be captured at all stages after harvest. In the context of the present invention, the image of each food product can be acquired using a number of computing devices, and a variety of illumination systems (halogen, LED, fluorescent and white lamps) to prevent any light interference.

At step 106, the captured image is analyzed by machine learning models to generate a QI (quality index) of the food product. The machine learning models may run on a processor in the computing device, or at a remote server communicatively coupled to the computing device. The analysis may include a first step of applying an image mask on the captured image, in that a specific image masking is applied to each image to remove non-interest elements (NIE) from the captured image. In an example, when the food product is strawberry, image masking removes all non-berry elements, and where relevant all berry based NIE. The analysis may further include a second step of transforming the image from an RGB base to CIELAB base to enhance image analysis, considering that the CIELAB color space is designed to provide a more consistent and accurate representation of color, which is generally preferred for human visual perception compared to RGB.

The analysis may further include a third step of entropy analysis, in that AI is applied to processed CIE images to calculate the entropy, standard deviation and range values of each image. This step involves the individual analysis of a series of individual attributes of the food product. The individual attributes include, but are not limited to, colour, calycs, shrivelling, decaying, bruising and physical damage and mould. Each individual attribute may be estimated based on visual assessment of the respective feature, using a vectoral algorithm. The average summation of each individual analysis of each individual attribute would generate the QI of the food product. At step 107, a future storage temperature at which the product is going to be stored or transported is inputted at a later time.

At step 108, the QI is merged with a variable order kinetic modelling database which converts the QI to a single RSL figure based on the future storage temperature. The term Spectral Residual Viability metric (SRV-m) is the technical term for the residual phrase and is representative of the SRV and corresponding RSL/marketability of the food (i.e. how much time is left before the product spoils). The term “metric” is assigned to represent the numeric quantification of the SRV and corresponding RSL.

At step 109, the captured image may be stored in a database for querying/retrieval at a later date.

In an embodiment of the present invention, the method 104 employs machine learning algorithms and product specific kinetics models for providing the end user with an SRV-m indicative of an accurate quality Index. The bespoke kinetic models are the results of laboratory trials to replicate the spoilage of fresh produce in a variety of environmental conditions replicating real life supply chain scenarios. These trials have been conducted in the environmental chamber laboratory and the produce has been graded (after a variety of exposure times to environmental stressors) using a trained panel. These samples have been photographed and used for training and validating the prediction accuracy of the application.

In an example, strawberries have been harvested and placed in punnets across 3 harvest days over a season. A sample of each has been stored at 1°, 10° and 20° C. for up to 11 days. For each harvest, samples have been analysed on harvest day (15 punnets) and at 2, 4, 7, 9, and 11 days (9 punnets for each temperature). Using both the captured images and the panel grading of the punnets, the two approaches have been used. One is development of regression models using selected features (IE) extracted from the images. The regression models have been developed to train the machine learning models by using the human graded images as a benchmark. Second is training CNN in a supervised manner where images of the strawberries have been input to the network and the quality of the strawberries have been the output of the network. The panel grading is where a panel of trained humans get to grade the berries (current industry standard). The human grades were recorded and used to grade the same berries with SpecRV to validate our system. After successful training, the CNN model has been able to correctly predict the quality of the strawberries.

The first approach includes the use of partial Least Squares Regression (PSLR) models using selected features (mean, median, range, entropy and standard deviation of RGB values and mean CIE Lab values) of masked strawberries images. The performance of the models has been evaluated using the root mean square error (RMSE) and the coefficient of determination (R²) of predicted vs measured strawberry appearance. The second approach includes CNN regression models developed to predict strawberry grading from images with varying success across training and validation stages with the most accurate yielding a RSME=0.31 and R2=0.88.

The method 104 may be employed independent of any individual operating system and would function across platforms to facilitate scaled deployment. The application executing the method 104 would facilitate processing on cloud/on edge and/or on chip depending on the commercial application.

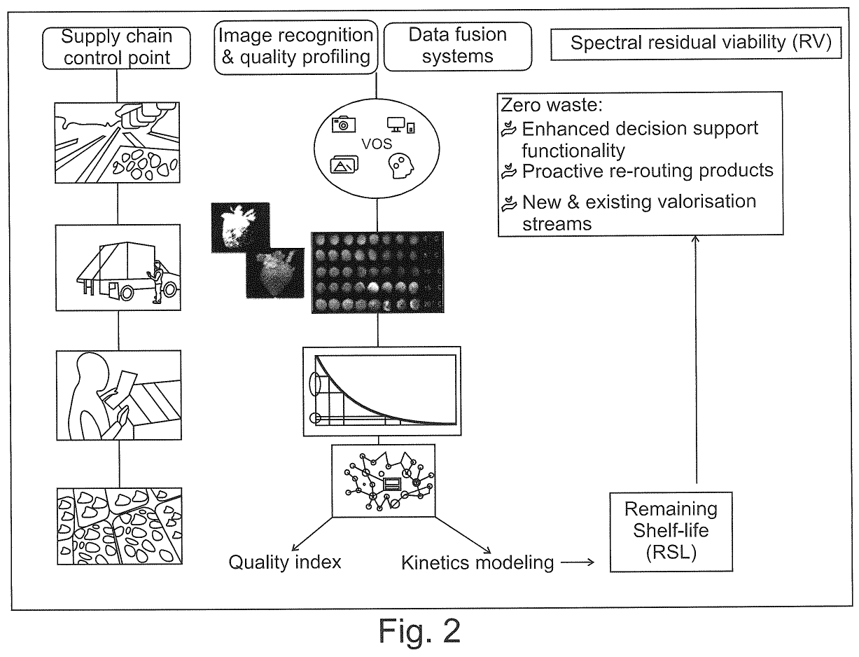

FIG. 2 illustrates the supply chain control points across which the SRV-m estimation can be applied to predict SRV and corresponding RSL allowing stakeholders to adopt a FEFO based biologic decision driven supply chain, as opposed to a FIFO decision-based system. The SRV-m would provide end users, irrespective of supply chain positioning, (1) enhanced decision support functionality and (2) ability to proactively re-route products and (3) identify new and existing valorisation streams. The SRV-m would provide an ability to make informed decisions and correctly manage the remaining steps of the supply chain to avoid loss or waste.

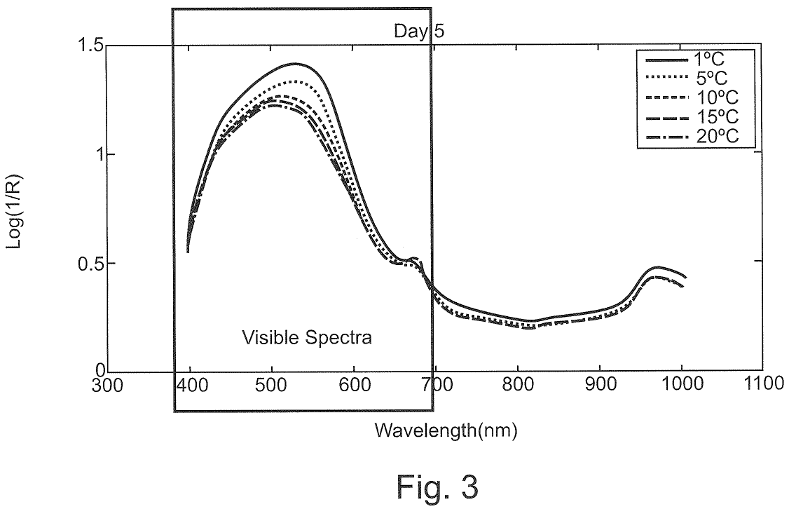

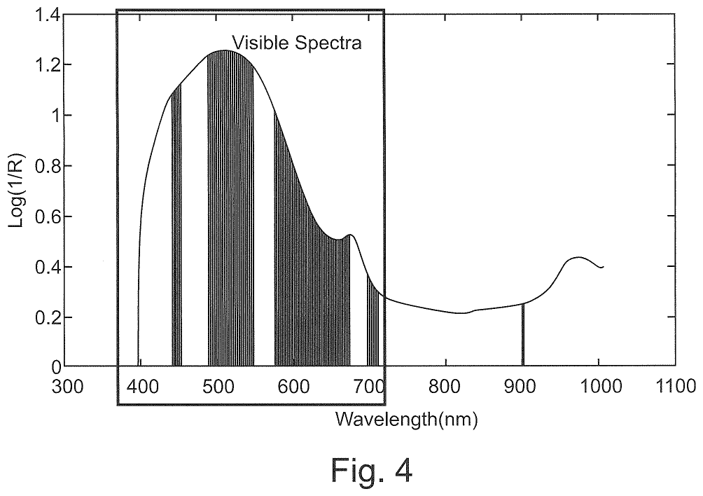

FIGS. 3 and 4 are examples of the spectral outputs where the visible spectra (enclosed in the box) represent the area of interest in the captured one or more images of strawberries. The peaks are representative of a number of biological indicators of the natural degradation process of fresh produce which is directly coupled with shelf life and consumer acceptability. This has facilitated a focus on the visible spectra to identify and map the biological spoilage process of fresh produce.

FIG. 4 represents a grouping of interest with respect to product freshness. The shaded groupings represent bands relevant to strawberry pigments (anthocyanins and chlorophyl) from a spectral image capture. These pigments are critical components in the current visual assessment of strawberries and fresh produce. FIGS. 3 and 4 establish that their acquisition and quantification is possible via visual spectral capture alone as the visible spectra of the strawberries stored in different conditions noticeably differ. This information can be correlated to shelf life and consumer acceptability (and proportional to waste and losses).

From FIG. 4, it is evident that the selected bands “of interest” for the appearance prediction model are concentrated in the area between 400-700 nm. The spectral area between 400-600 nm is associated with strawberry pigments namely anthocyanins and chlorophyl. The area between 696-708 nm may be attributed to chlorophyl a but has also been associated with carbohydrate absorption and the 4th C—H and 3rd O—H in carbohydrates.

These captured changes (peak clusters of FIG. 4) in colour remain to be amongst the main visual characteristics determining quality perception and indicating ripening/overripening. Finally, it is also evident that the spectral bands between 525-692 nm contain biologic critical information relating to the colour attributes relating to the overall visible image capture.

Spec-RV would provide monetizable alternatives to existing operational processes. Once waste has been identified, the produce is already damaged and there are few alternatives for re-purposing of product. Spec-RV provides an ability to identify imminent waste thus allowing for actions to prevent waste during transport or further storage. This technology can be adopted across a variety of fresh produce supply chains such as lettuce, berries and an array of fast-moving consumer goods. Although, the Spec-RV estimation models has been illustrated for strawberries, it would be apparent to one of ordinary skill in the art, that the Spec-RV estimation models may be designed for various other food products.

Spec-RV is estimated by creating a cyber-physical ecosystem consisting of Vision Optical Systems (VoS), sophisticated deep learning architectures (CNNs), followed by a combination of vectoral machine learning algorithms and kinetic models tasked with reducing food losses and waste through calculating the SRV and corresponding RSL of food groups.

Spec-RV estimation models have potential for all fresh produce and can be adapted (Quality index and CNN specific) to any type of produce. Spec-RV has the ability to be adapted to all produce supply chains including leafy vegetables and berries. Spec-RV would provide the end user with an RSL computation which may then be used to action shipments to suitable products based on time and location relative to RSL thus reducing product loss and/or waste. The Spec-RV estimation method provides more efficient operational control and enhanced empowerment for all chain actors, in a plurality of ways including:
1. Farm level (digital state of product in real time to enhance revalorisation potential)
2. Wholesale level (digital grading used to predict routing and margins)
3. Retail (best before replacement, more frequent stock rotations thus less waste)
4. Consumer (more informed decision-making and less impulse buying) leading to excess household waste

Spec-RV would adopt “Biologic” based decisions at all stages across the supply chain as opposed to process decisions i.e., “Can we ship this product without it spoiling?” as opposed to “we have to ship 5 tonnes today so just do it—it may not go bad”.

Spec-RV would transform existing subjective processes of estimating remaining shelf-life for a “Biologic based decision framework”, replacing the FIFO approach with a digitised FEFO approach, transforming global supply chains, minimising food loss/waste and underpinning circular bioeconomic opportunities from food waste. Spec-RV would also allow stakeholders to identify more precisely when a product should be removed from “general open shelf presentation” and sent for commercial repurposing. Spec-RV would eventually replace existing “Best Before” and “use by” dates which are a key cause of unnecessary food waste at retail thus reducing the cost of food waste and facilitating more efficient logistics management through the entire logistics system allowing all stakeholders to be more waste conscious. Spec-RV would be deployable across the full agri-food chain and would require little to no user training which will increase its chances of adoption. Spec-RV interface would be a cross platform system and would not require any level of literacy for the user. The proposed system would facilitate the placement of higher quality product on retail shelves thus delivering safer and more nutritious food to the consumer, facilitating a more food secure society and a happier and more loyal customer. Further, the Spec-RV estimation would be independent of language and stakeholder engagement, thereby strongly encouraging the use of icons and animations as opposed to text.

In the specification the terms “comprise, comprises, comprised and comprising” or any variation thereof and the terms include, includes, included and including” or any variation thereof are considered to be interchangeable, and they should all be afforded the widest possible interpretation and vice versa.

The invention is not limited to the embodiments hereinbefore described but may be varied in both construction and detail.

1. A method for assessing spectral residual viability (SRV) and corresponding remaining shelf life (RSL) of a food product, comprising:
capturing one or more images of the food product by a camera;
analysing the one or more images to perform an assessment of a plurality of individual interest elements (IE) of the food product, using respective plurality of vectoral algorithms, wherein IE are generated by removing non-interest elements (NIE) from total elements (TE) of the food product;
estimating a Quality Index (QI) of the food product based on an average summation of each individual attribute, wherein each individual attribute is estimated based on one or more IE of the image;
receiving a future storage temperature at which the food product is going to be stored or transported at a later time; and
estimating the SRV and corresponding RSL of the food product based on the QI and the future storage temperature.

2. The method as claimed in claim 1, wherein the analysing an image comprises:
applying an image mask on the image to remove NIE from TE of the food product;
transforming the image from an RGB base to CIELAB base to enhance IE discrimination potential for image analysis, and accurately represent the perceived colour of images being analysed; and
using machine learning models to estimate an entropy, standard deviation and range values of the image for a plurality of individual attributes of the food product.

3. The method as claimed in claim 1, wherein each individual attribute is selected from colour, calyx wilting, shrivelling, bruising and physical damage, decay and mould.

4. The method as claimed in claim 1 further comprising:
employing machine learning algorithms and product specific kinetics modelling providing the end user with a product specific fully digitized SRV and corresponding RSL based on expected storage conditions, and time/distance to final destination.

5. The method as claimed in claim 1 further comprising estimating the QI of the food product based on analysis of visual spectra of wavelength ranging from 380 nm to 750 nm and frequency ranging from 4 Hz to 7.5*10¹⁴ Hz of the one or more captured images.

6. The method as claimed in claim 1 further comprising merging the QI with a variable order kinetic modelling database to generate the SRV and corresponding RSL based on the future storage temperature.

7. The method as claimed in claim 1 further comprising storing the one or more captured and analysed images in a database for querying/retrieval at a later date.

8. A system for assessing spectral residual viability (SRV) and corresponding remaining shelf life (RSL) of a food product, comprising:
a memory to store the one or more instructions;
a processor to execute the one or more instructions to:
capture one or more images of the food product by a camera;
analyse the one or more images to perform an assessment of a plurality of individual interest elements (IE) of the food product, using respective plurality of vectoral algorithms, wherein IE are generated by removing non-interest elements (NIE) from total elements (TE) of the food product;
estimate a Quality Index (QI) of the food product based on an average summation of each individual attribute, wherein each individual attribute is estimated based on one or more IE of the image;
receive a future storage temperature at which the food product is going to be stored or transported at a later time; and
estimate the SRV and corresponding RSL of the food product based on the QI and the future storage temperature.

9. The system as claimed in claim 8, wherein the analysing the image comprises:
applying an image mask on the image to remove NIE from TE;
transforming the image from an RGB base to CIELAB base to enhance (IE) discrimination potential for image analysis, and accurately represent the perceived colour of images being analysed;
using machine learning models to estimate an entropy, standard deviation and range values of the image for a plurality of individual attributes of the food product.

10. The system as claimed in claim 8, wherein each individual IE attribute is selected from colour, calyx wilting, shrivelling, bruising and physical damage, decay and mould.

11. The system as claimed in claim 8, wherein the processor is further configured to:
employ machine learning algorithms and product specific kinetics modelling providing the end user with a product specific fully digitized SRV and corresponding RSL based on expected storage conditions, and time/distance to final destination.

12. The system as claimed in claim 8, wherein the processor is further configured to estimate the QI of the food product based on analysis of visual spectra of wavelength ranging from 380 nm to 750 nm and frequency ranging from 4 Hz to 7.5*10¹⁴ Hz of the one or more captured images.

13. The system as claimed in claim 8, wherein the processor is further configured to merge the QI with a variable order kinetic modelling database to generate the SRV and corresponding RSL based on the future storage temperature.

14. The system as claimed in claim 8, wherein the processor is further configured to store the one or more captured and analysed images in a database for querying/retrieval at a later date.
